(19) United States
(12) Patent Application Publication
(10) Pub. No.: US 2023/0390476 A1
(43) Pub. Date: Dec. 7, 2023

(54) IMPROVED BODY DRAINAGE APPARATUS

(71) Applicant: Thoragen AB, Solna (SE)

(72) Inventor: Magnus EMMOTH, Vaxholm (SE)

(73) Assignee: Thoragen AB, Solna (SE)

(21) Appl. No.: 18/248,717

(22) PCT Filed: Oct. 19, 2021
(86) PCT No.: PCT/EP2021/078944

§ 371 Date: Apr. 12, 2023

(30) Foreign Application Priority Data
Oct. 19, 2020 (SE) 2051209-1

(51) Int. Cl. A61M 1/00

(52) U.S. Cl. CPC A61M 1/73; A61M 2205/502

(57) ABSTRACT

A drainage apparatus for the drainage of bodily fluids and air from a patient, wherein the drainage apparatus comprises means for generating a suction pressure (10, 430), and means for monitoring drainage parameters (415) including suction pressure, amount of drained fluid, and amount of air leakage. A peristaltic pump (10, 430) is encapsulated in a housing (11), and a detachable electronic fluid collection unit (15). Said collection unit (15) is provided with a disposable sensor module (5) having means for detecting air leakage (6) and pressure alterations (8), and a storage memory for the purpose of data storage (17) connected between the inlet of the collection unit and the peristaltic mechanism by a flexible tubing (4). Said pump housing (11) being provided with a pressure sensor (13) to control a strength of suction in relation to delta pressure during patient respiration (22), and a pressure sensor (20) for the purpose of utilizing atmospheric pressure as reference. The amount of bodily fluid drained will be measured by a disposable capacitive fluid level sensor (14), separated from the pump housing (11). Said collection unit (15) being detachable and being supported by a portable battery-powered data logging unit (21) during mobilization of the patient.

This application claims priority under 35 USC 119(a)-(d) to SE patent application No. 2051209-1, which was filed on Oct. 19, 2020, the entire contents of which are incorporated herein by reference.

### TECHNICAL FIELD

The present invention relates generally to a drainage apparatus and a method for drainage of excess body fluid from a body cavity of a patient. In particular, the present invention relates to such for draining of excess fluid from the pleural cavity of a patient.

### BACKGROUND

In contemporary medical care, the movement of fluid from a body cavity to another point for collection is a routine need and can be performed in a number of ways. When tubing is used for carrying the fluid during the movement either gravity or a pump is utilized to create and/or sustain a suction pressure needed to move the fluid from one point to another.

In lung medicine, when recovering from pneumothorax, i.e., a collapsed lung due to e.g. lung surgery, suction pressure may be applied by inserting a chest tube between the ribs to remove excess air in the space between the chest wall and the lung.

### SUMMARY OF THE INVENTION

At times, the movement of fluid from the body must be performed in a gentle, slow and steady manner. Such gentle, slow and steady manner can be designated “peristalsis”. Peristaltic pumping may be performed in a number of ways including, but not limited to, hand pumping or use of an electrically driven peristaltic pump.

Additional known methods for drainage procedures include use of occurrences such as plastic vacuum suction bottles, wall suction and portable suction pumps. These methods typically produce a constant suction rather than a peristaltic suction. These methods also include plastic bottles that are pre-assembled with a vacuumed pre-set under pressure causing inadequate suction; are bulky and causing storage, operational and shipping difficulties; typically, are limited in size necessitating frequent changes during the procedure; require special medical waste handling procedures; and when shattered in use create the danger of contamination problem of body fluids. Wall suction, in addition to providing only constant suction, is not readily available in all clinical settings. Wall units tend to create greater suction forces than what is safe for a normal drainage procedure.

The objective of the present invention is to provide an improved device and an improved method for draining fluid from the pleural cavity of a patient. The improvement may lay in shorter healing time, less pain, easier handling or all of the above.

Generally, there is provided an apparatus and a method for draining and collection of a bodily fluid, the apparatus comprises a peristaltic pump device for creating a drainage suction pressure, a control unit for controlling the magnitude of the suction pressure by controlling the speed of the pump, based on signals from one or more sensors including one or more pleural pressure sensors. A collection container for collection the bodily drainage fluid may also be included.

The corresponding method comprises steps for the regulation and control of the peristaltic pump movements and step(s) for collection of the bodily drainage fluid.

According to a first aspect, there is provided an apparatus for aspirating drainage fluid from a body cavity, the drainage system comprising:
a peristaltic pump 10 for generating a suction pressure for aspirating drainage fluid from a patient;
a fluid collection unit 15 provided for collecting fluid from the patient and for measuring volume of the collected fluid
a separator unit 3 arranged to separate air from bodily fluid arriving via tubing 1 to the collection unit 15, and
   wherein the peristaltic pump is configured to be connected to the patient and to the collection unit via appropriate tubing,
   and wherein
a suction pressure sensor is configured to sense the pressure in an intrapleural space;
a processor is electrically connected to the suction pressure sensor, and to the pump, and configured to continuously collect pressure data from the pressure sensor, and wherein the processor is configured to repeatedly calculate, based on collected pressure data, a minimum or inhalation pressure encountered during inhalation, representing a mean value of the minimum intrapleural pressure at patient inhalation of two or more consecutive inhalations, and
   wherein the processor also is configured to repeatedly calculate, based on collected pressure data, a maximum or exhalation pressure, representing a mean value of the maximum intrapleural pressure at patient exhalation of two or more consecutive exhalations, and wherein the drainage system is configured to present or otherwise communicate the inhalation pressure and/or the exhalation pressure or signals or values being calculated from them. In particular, the apparatus is configured to calculate a Delta P pressure which is calculated as a difference between the exhalation and inhalation intrapleural pressures. The apparatus may further comprise one or more sensors for measurement and display of drained bodily fluid volume and detection of potential air leakage from the damaged lung tissue.

Furthermore, the device is configured to be able to provide a method to measure the lung expansion by the utilization of delta pressure during the respiration cycle.

The apparatus may further include a decision support system, capable of suggesting a diagnosis and/or a prognosis of the illness of the patient.

Thus, there is provided a drainage apparatus for aspirating and measuring of body fluids, the apparatus comprising a peristaltic pump device for a pressure controlled peristaltic movement regulation of fluid transportation comprising: a peristaltic pump housing and a peristaltic mechanism unit arranged in the peristaltic housing and a fluid collection unit being able to be secured releasable on the peristaltic pump housing. The drainage apparatus is arranged to drain the fluid through tubing connected to the patient and said tubing is in a distal end connected to a collection unit that is arranged in the direction of fluid. The inside of the collection unit stands in contact with a pressure sensor located near the collection unit for the purpose of measuring the pressure and supply pressure readings to the processor for controlling the peristaltic pumping mechanism by comparing the current suction pressure with a pre-set desired suction pressure and pause pumping if the desired suction pressure is reached. Further, the processor is configured to estimate the amount of air-leakage by using readings from a disposable flow-sensor module. As an alternative to engage the flow sensor for air leakage detection the system could as a complement utilize the pressure sensor in the sensor module 5 in combination with the fluid level sensor 14 in the collection unit 15 whereas the loss of pressure over time indicates the volume of air entering the collection unit 15 from the bodily cavity. The collection unit is arranged to have a fixed known volume to facilitate volume calculations.

According to a second aspect there is provided a collection unit for collecting drainage fluid from a patient's body with the aid of a suction pressure, the collection unit comprises:
a container made of a pressure tight material, and rigid enough not to buckle when submitted to the suction pressure;
an inlet opening; configured for connecting the collection unit to a patient's body in need of drainage;
an outlet opening, configured to be connected to a source of suction pressure;
   The collection unit is provided with an arrangement of multiple disposable screen-printed capacitive filling-level sensors arranged on an outside face of the collection unit, and on the side facing the pump housing. The filling level sensors comprises a self adhesive conducting film with printed areas that are connected or not connected with each other. The conductive film may be based on an aluminium film, or a cupper film, or a carbon based film, or on a silver film. Most preferred is an aluminium film because it has proven, during tests, to be most reliable and easy to manufacture and adjust to this use.

The metal film comprises at least one, preferably three electrically separated elongated areas extending from the bottom of the collection unit and up to a maximum filling level of the collection unit. Each elongated area extend further to a connector area, which connector area is arranged to adhere to a toot-like springy portion of a polymer frame.

The multiple disposable screen-printed capacitive filling-level sensors are provided for the purpose of detecting a filling level in the fluid collection unit. The capacitive filing sensors may also comprise a connector for connecting an electrical cable that transfers the capacitive signals from the bag to the processor. The fluid level sensor connector on the collection unit provides an element with a multiple of spring-loaded connectors being able to secure a safe connection between the sensor and the counterpart being the receiving connector for the signal from the fluid measurement.

The collection unit may further be provided with an accelerometer to sense the direction of gravity relatively to the axes of the collection unit, in order to issue an alarm should the collection unit be tilted unacceptably much, making measurements of filling level erroneous or meaningless.

According to a third aspect, there is provided a method utilizing a pressure sensor as means for detecting the patients respiratory rate and thereof related changes in the intrapleural pressure during inhalation, expiration and lung re-expansion. The pressure variation during the respiration phase and during the lung expansion is decreasing in linearity to the lung expansion. The pressure variation being a marker for lung expansion or lung deflation.

Further, there is provided means and methods for measuring intrapleural pressure and to calculate and display a pressure difference between intrapleural pressure at inhalation, and intrapleural pressure at exhalation.

Still further, the apparatus may be provided with an automatic function, or artificial intelligence, if it is preferred to designate it that way, to adjust the suction pressure automatically, based on changes in the pressure difference described above. In a typical case, suction pressure will be adjusted in steps towards less suction as pressure difference decreases with healing.

Further, the apparatus may be provided with a pressure adjustment function, capable of adjusting a pressure difference between two pressure sensors arranged to sense intrapleural pressure.

### BRIEF DESCRIPTION OF THE DRAWINGS

In order that the manner in which the above recited and other advantages and objects of the invention are obtained will be readily understood, a more particular description of the invention briefly described above will be rendered by reference to specific embodiments thereof which are illustrated in the appended drawings.

Understanding that these drawings depict only typical embodiments of the invention and are not therefore to be considered to be limiting of its scope, the invention will be described and explained with additional specificity and detail through the use of the accompanying drawings in which:

FIG. 1 shows a schematic view of a drainage system according to an embodiment of the invention.

FIG. 2a shows a schematic front view of normal lungs.

FIG. 2b shows a schematic front view of a normal right lung and a collapsed left lung.

FIGS. 3a, 3b, and 3c shows a connector plate for electrical connection between the fluid level sensor of a collection unit and a receiving connector of a pump housing.

FIG. 4 shows a block diagram of a first drainage apparatus.

FIG. 5 shows a block diagram of a second drainage apparatus.

FIG. 6 shows a flowchart of a first method to determine a pressure difference.

FIG. 7 shows a flowchart of a second method to determine a pressure difference.

FIGS. 8a and 8b shows intrapleural pressure signal variation during inhalation and exhalation during respiration of a simulated human lung during a sped-up healing process.

### DETAILED DESCRIPTION

During the remainder of this document the following words and abbreviations will be used with their respective meanings.
Minimum, min: The lowest value of something, sometimes within a specified area or interval. In this respect a (negative) pressure of −15 (minus 15) is lower than a pressure of −10 (minus 10)
Maximum, max: The highest value of something, sometimes within a specified area or interval. In this respect a (negative) pressure of −5 (minus 5) is higher than a pressure of −10 (minus 10)
Intrapleural Pressure: The pressure in the space between the lung and the chest wall. This pressure is usually negative, i.e., lower than the atmospheric pressure to keep the lung to adhere to the chest wall. The intrapleural pressure also usually varies with diaphragmic and rib cage movements during respiration.
Respiration: The activity of breathing. Involves inhalation and exhalation
Delta P: In this context, Delta P is used to denote the pressure difference between a maximum and a minimum intrapleural pressure.
Active mode: A mode of a device wherein the device is actively performing or producing something, such as a pump producing a suction pressure.

FIG. 1 shows an embodiment of a drainage system for draining excess body fluid from a patient. A microcontroller unit is arranged and configured to, in conjunction with the pump 10, regulate an rpm of the pump to maintain a set suction pressure or otherwise determined suction pressure at the patient and/or in a reservoir or collection unit 15.

The drainage system may further be configured to continually measure pressures at one or more specific locations, and to store, display and/or use measured pressure values in order to convey adequate information to responsible personnel, in order provide basis for a manual or automated adjustment of the suction pressure throughout the healing process.

In an active mode the pump 10 is configured to suck fluid and air from the patient's bodily cavity and when this air/fluid mixture reaches the inlet of the collection unit 15, the air is separated from the fluid by a separation unit 3 and the air is then guided through a filter 7. From the filter 7 the air is led via a flexible tube 4, directed by the pump 10 to a sensor module 5.

The fluid is thus dumped into the collection unit 15. A fluid level sensor 14 is arranged to measure a fluid level in the collection unit 15.

Sensors are provided to sense drainage data. A processor and a memory unit are arranged to log drainage data, and to process and interpret drainage data. Pre-set values are recognized and are used by the processor to control the rotation rate of the pump 10 in order to maintain a pressure, such as an intrapleural pressure or a pressure in the collection unit 15.

Control parameters handled by the processor may include the following; pressure (mm H2O, fluid volume (ml) and air leakage (ml/min). In the case the pump 10 is not active and air needs to be released from the collection unit 15, the air is released to the atmosphere via the positive relief valve 9. Air evacuation from the collection unit 15 during an active mode is released through the atmosphere via the non-return-valve 12 being provided with a filter to prevent the spread of viruses and bacteria into a hospital or other environment. The filter of the non-return valve 12 is a filter capable of capturing 99.95% of virus size particles, e.g. a so called Hepa filter.

Intrapleural Pressure Signal

An intrapleural pressure is propagated from a chest tube 101 inserted in the intrapleural space, via a flexible tube 2 to a reference pressure sensor 13 to facilitate measurement of the intrapleural pressure. The chest tube is also connected to the collection unit such that fluid can be drained. The reference pressure sensor 13 is connected to the processor and provides an intrapleural pressure signal representing the fluctuating pressure in the intrapleural space. The intrapleural pressure varies with each breath and also with progress of a healing process.

Delta Pressure

The pressure in the sensor module 5 is cross-referenced to a reference pressure sensor 13 in the pump housing 11 and to a pressure sensor as reference to the atmospheric pressure 20. The reference pressure sensor 13 is monitoring the respiratory rate and the Delta pressure between inhalation and exhalation. Said reference pressure sensor 13 sends information to the processor to enable the processor to display and/or regulate strength of suction pressure in relation to the Delta Pressure.

Operating Modes

The system may be featured by pre-settings whereas the operator selects one suitable operation mode for the clinical situation. Said settings can be adjusted by an administrator.

Decision Support System

The drainage apparatus may be provided with a decision support system. The decision support system may comprise a separate processor or may be software programmed into a microcontroller of the drainage apparatus. The decision support system is configured to collect consecutive sensor values over time and to calculate values that can be presented as diagrams or that can be utilised to present decision parameters to physicians or other personnel operating the drainage apparatus. The drainage apparatus may also be provided with wireless or wired communication capabilities for sending decision support data to a remote location.

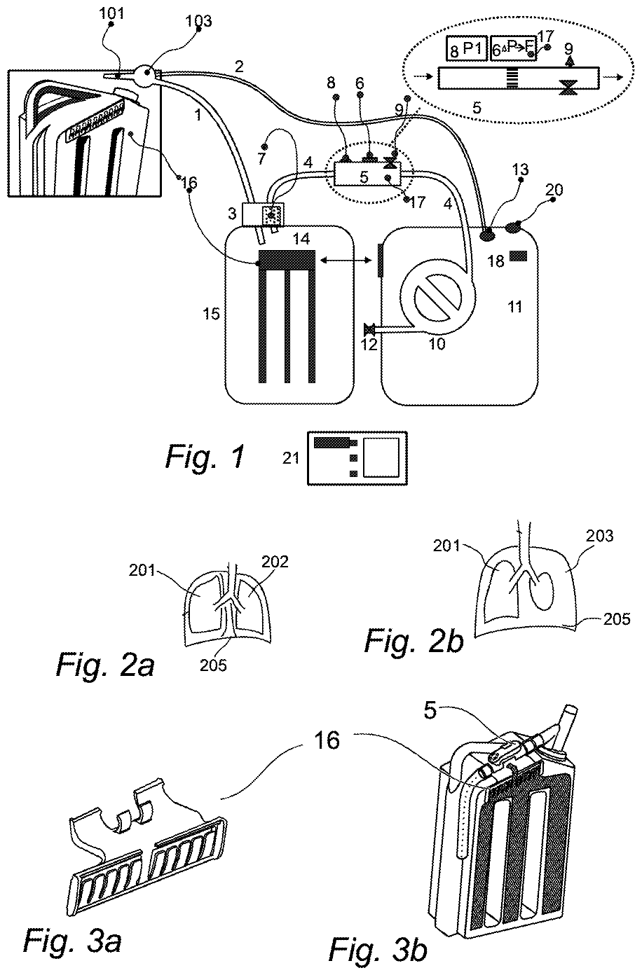

FIG. 2b shows a schematic front view of a normal right lung 201 and a collapsed left lung 203. One of the objects of the present invention is to provide a device for improving healing, and decreasing recovery time to restore a collapsed lung, also called pneumothorax, to a normal, un-collapsed condition. By controlled suction and drainage of air and liquid of the pleural space, the lung expands and the pleural space returns to a condition without air, as seen in the pleural space 205 of the right lung 202 (FIG. 2a).

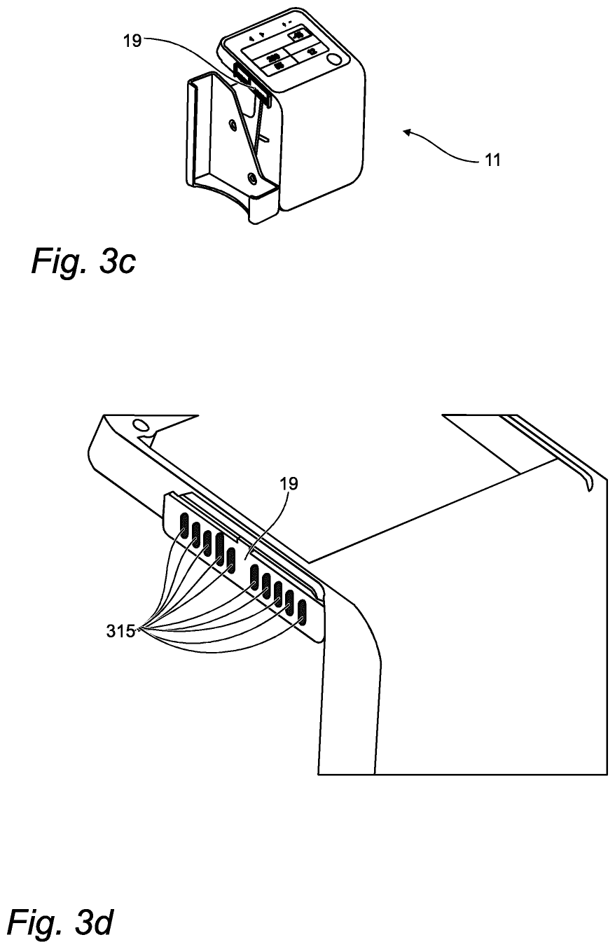

FIGS. 3a, 3b, and 3c show an embodiment of a connector plate 16 provided with spring-loaded projections enabling a firm contact between the fluid level sensor 14 and the receiving connector 19 in a pump housing 11. The connector plate 16 also serves to hold the sensor module 5 in position. Thus, the collection unit 15 is provided with an arrangement of multiple disposable screen-printed capacitive filling-level sensors arranged on an outside face of the collection unit, and on the side facing the pump housing. The filling level sensors comprises a self-adhesive conducting film with printed areas that are connected or not connected with each other. The conductive film may be based on an aluminium film, or a cupper film, or a carbon based film, or on a silver film. Most preferred is an aluminium film because it has proven, during tests, to be most reliable and easy to manufacture and adjust to this use.

The metal film comprises at least one, preferably three electrically separated elongated areas extending from the bottom of the collection unit and up to a maximum filling level of the collection unit. Each elongated area extends further to a connector area, which connector area is arranged to adhere to a toot-like springy portion of a polymer frame.

The multiple disposable screen-printed capacitive filling-level sensors are provided for the purpose of detecting a filling level in the fluid collection unit. The capacitive filing sensors may also comprise a connector for connecting an electrical cable to transfer capacitive signals from the collection unit 15 to a processor. The fluid level sensor connector 16 of the collection unit 15 provides an element with a multiple of spring-loaded connectors being able to secure a safe connection between the sensor 14 and the counterpart being the receiving connector 19 for the signal from the fluid measurement.

FIG. 3d shows a detail of an upper portion of the pump housing 11 showing a receiving connector 19 arranged to engage and make electrical contact between portions of the metal film of the collector unit 15 and contact pads 315 of the receiving connector 19 of the pump housing 11. The connector pads 315 are made for repeated use and are made of metal, while the spring loaded connector of the collection unit are made for single use only and are provided with the mentioned metal film, which can be made relatively thin to achieve low price and environmental friendliness.

Further, the volume of air entering the collection unit 15 may be determined with the aid of a pressure sensor arranged in the sensor module 5, and the processor is configured to update the known volume of the dead space in the collection unit 15 continuously by reading the fluid level sensor 14. By measuring the time for a change in pressure in the collection unit 15 with a known volume of the dead space with the aid of a fluid level sensor 14 the processor may calculate the volume of air.

Double Lumen Tube System

The drainage apparatus may preferably be provided with a double lumen tube system. A double lumen catheter may be arranged between the patient and the collection unit in order to simplify handling and reduce risk of tangling. The double lumen catheter provides a first lumen for transporting fluid from the patient to the collection unit, and a second lumen, extending further than the first lumen, constituting a measure connected to the reference pressure sensor 13 and to the pressure adjustment valve 103.

Pressure Adjustment Function

The drainage apparatus may further be configured to comprise a pressure adjustment function. An electrically operated first valve, which may be the pressure adjustment valve 103, is arranged to temporarily open a connection to ambient air in the reference tube 2 to let air rush to the point where the reference tube 2 and the drainage tube 1 meet. The first valve is electrically connected to the processor such that the processor can control actuation, i.e., opening and closing of the first valve. The processor may preferably open the first valve at regular intervals such as once every five minutes for typically one millisecond. Thus, the open period of the first valve is arranged to be relatively short, the amount of air limited, and suction pressure adjusted, such that there is minimal risk of causing pain to the patient or of delaying healing. The system may be configured to open the first valve at a pressure lower than −40 mbar to act as a safety valve.

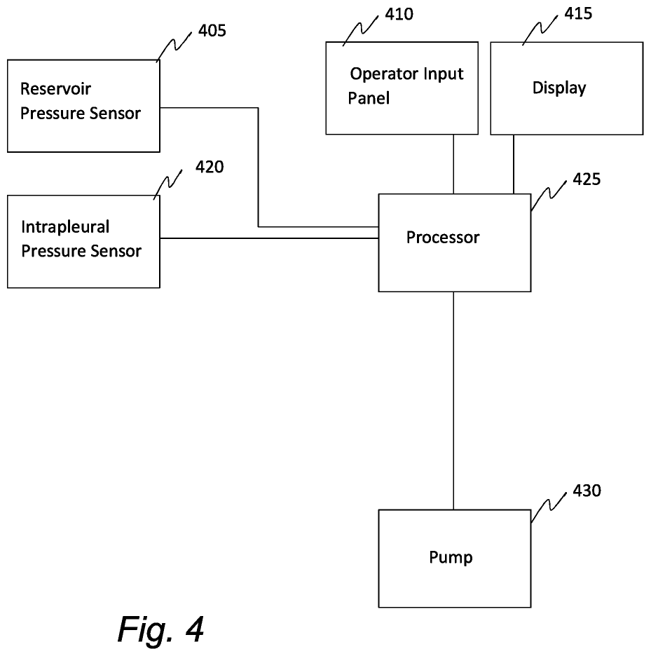

FIG. 4 shows a block diagram of a drainage apparatus. An intrapleural pressure sensor 420 is arranged to measure an intrapleural pressure in the space between a lung and a thoracic wall of a patient. The intrapleural pressure sensor 420 is connected to a processor 425 to convey an intrapleural pressure signal to the processor. The processor is configured to process the intrapleural pressure signal to determine a Delta P pressure signal, see below, which Delta P pressure signal may be displayed on a display 415 connected to the processor 425.

Further, an operator input panel 410 may be arranged to facilitate operator inputs, such as settings, to the processor 425. The processor may further be provided with a memory (not shown).

The processor may further be connected to a pump 430, such as a peristaltic pump 10, for generating a suction pressure, that may be propagated to the patient via a reservoir. The reservoir may be provided with a reservoir pressure sensor 405 that may sense a reservoir pressure in the reservoir and produce a reservoir pressure signal. The sensor may be connected to the processor 425 to convey the reservoir pressure signal to the processor 425.

The processor may further be arranged to adjust the set suction pressure automatically, based on changes of the Delta P pressure.

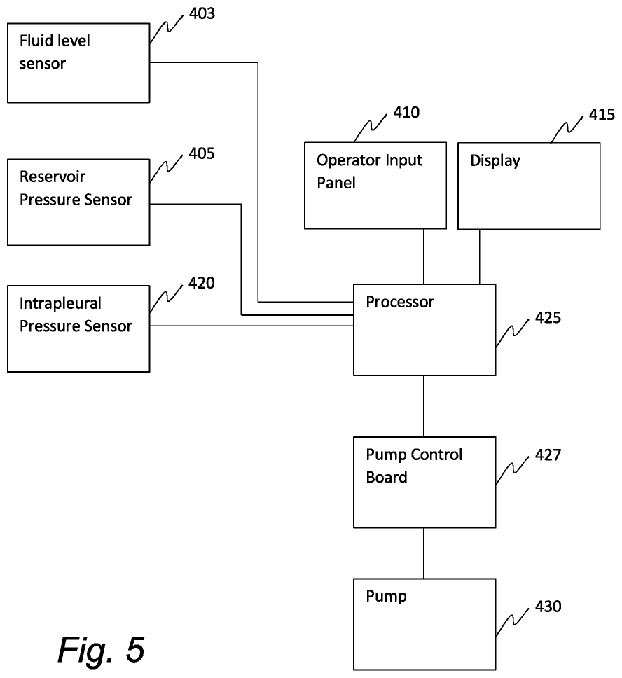

FIG. 5 shows a block diagram of a further drainage apparatus. An intrapleural pressure sensor 420 is arranged to measure an intrapleural pressure in the space between a lung and a thoracic wall of a patient. The intrapleural pressure sensor 420 is connected to a processor 425 to convey an intrapleural pressure signal to the processor. The processor is configured to process the intrapleural pressure signal to determine a Delta P pressure signal, see below, which Delta P pressure signal may be displayed on a display 415 connected to the processor 425.

Further, an operator input panel 410 may be arranged to facilitate operator inputs, such as settings, to the processor 425. The processor may further be provided with a memory (not shown).

The processor may further be connected to a pump 430, such as a peristaltic pump 10, for generating a suction pressure, that may be propagated to the patient via a reservoir. The reservoir may be provided with a reservoir pressure sensor 405 that may sense a reservoir pressure in the reservoir and produce a reservoir pressure signal. The sensor may be connected to the processor 425 to convey the reservoir pressure signal to the processor 425.

The reservoir is configured to collect fluid drained from the patient. The reservoir is to this end provided with a fluid level sensor 403, 14, and the fluid level sensor produces a fluid level signal, and is electrically connected to the processor to bring the fluid level signal to the processor. The processor may process the fluid level signal before presenting it on the display 415.

The Processor and the pump may be connected via a pump control board in order to facilitate control of the pump using appropriate power electronics to amplify control signals from the processor.

The processor may further be arranged to adjust the set suction pressure automatically, based on changes of the Delta P pressure.

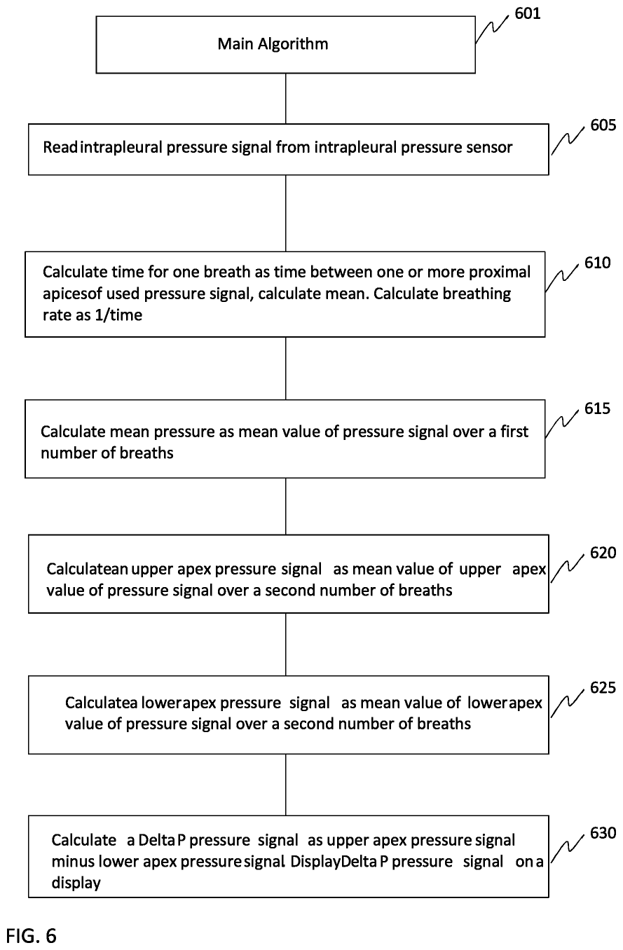

FIG. 6 shows a flowchart of a first method to determine a pressure difference. The method comprises the following steps:
Read 605 an intrapleural pressure signal from an intrapleural pressure sensor.
Calculate 610 a single breath time for one breath as time between a first and a second apex 801, 803, of the intrapleural pressure signal. Calculate respiratory rate RR as 1/single breath time. The calculations may result in more stable values if a mean value is calculated over two or more breaths.
Calculate 615 mean intrapleural pressure as mean value of intrapleural pressure signal over a first number of breaths.
Calculate 620 an upper apex pressure signal as mean value of upper apex value 801, 803, 805 of intrapleural pressure signal over a second number of breaths or, over first a pre-set length of time, such as e.g. 60 second;
Calculate 625 a lower apex pressure signal as mean value of lower apex value 802, 804, 806 of intrapleural pressure signal over a third number of breaths or, over first a pre-set length of time, such as e.g. 60 seconds
Calculate a Delta P pressure signal as upper apex pressure signal minus lower apex pressure signal. Delta P pressure may be displayed on a display 415.

The method may further include the step of adjusting the set suction pressure automatically, based on changes of the Delta P pressure.

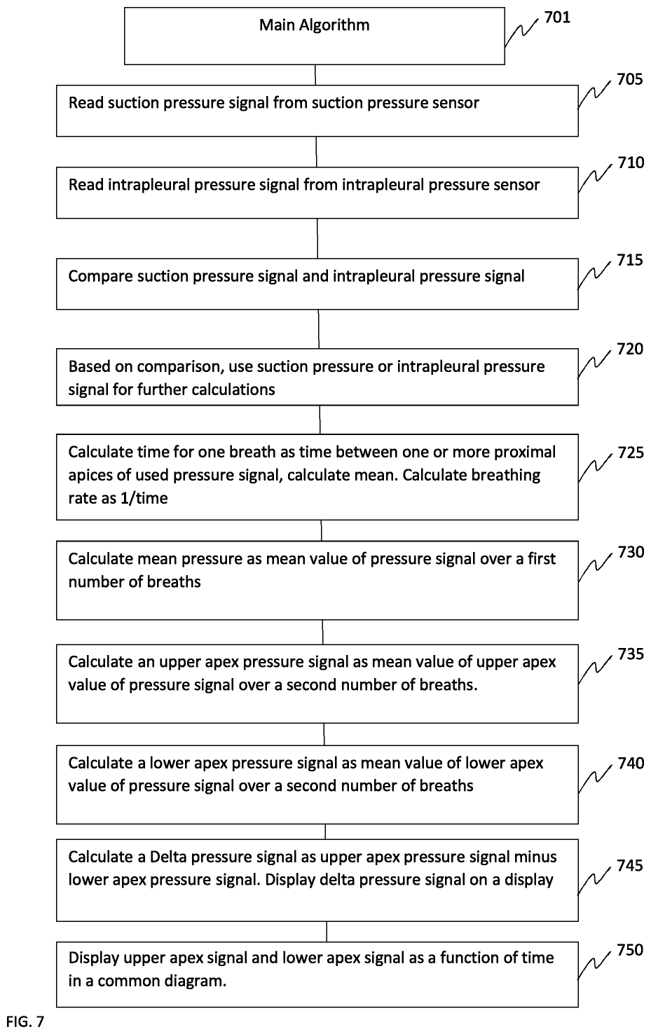

FIG. 7 shows a flowchart of an augmented method to determine a pressure difference. The method comprises the following steps:
Read a suction pressure signal from a first suction pressure sensor, the first suction pressure sensor may be a reservoir suction pressure sensor arranged to measure the pressure in the reservoir, also known as the collection unit 15
Read 710 an intrapleural pressure signal from an intrapleural pressure sensor;
Compare 715 the suction pressure signal and the intrapleural pressure signal;
Based 720 on the comparison, use suction pressure or intrapleural pressure signal for further calculations;
Calculate 725 a single breath time for one breath as time between a first and a second apex 801, 803, of the used pressure signal. Calculate respiratory rate RR as 1/single breath time. The calculations may result in more stable values if a mean value is calculated over two or more breaths.
Calculate 730 mean intrapleural pressure as mean value of used pressure signal over a first number of breaths.
Calculate 735 an upper apex pressure signal as mean value of upper apex value 801, 803, 805 of intrapleural pressure signal over a second number of breaths or, over first a pre-set length of time, such as e.g. 60 second;
Calculate 740 a lower apex pressure signal as mean value of lower apex value 802, 804, 806 of used pressure signal over a third number of breaths or, over first a pre-set length of time, such as e.g. 60 seconds
Calculate a Delta P pressure signal as upper apex pressure signal minus lower apex pressure signal. Delta P pressure may be displayed on a display 415;
Display upper apex signal and lower apex signal as a function of time in a common diagram.

The method may further include the step of adjusting the set suction pressure automatically, based on changes of the Delta P pressure.

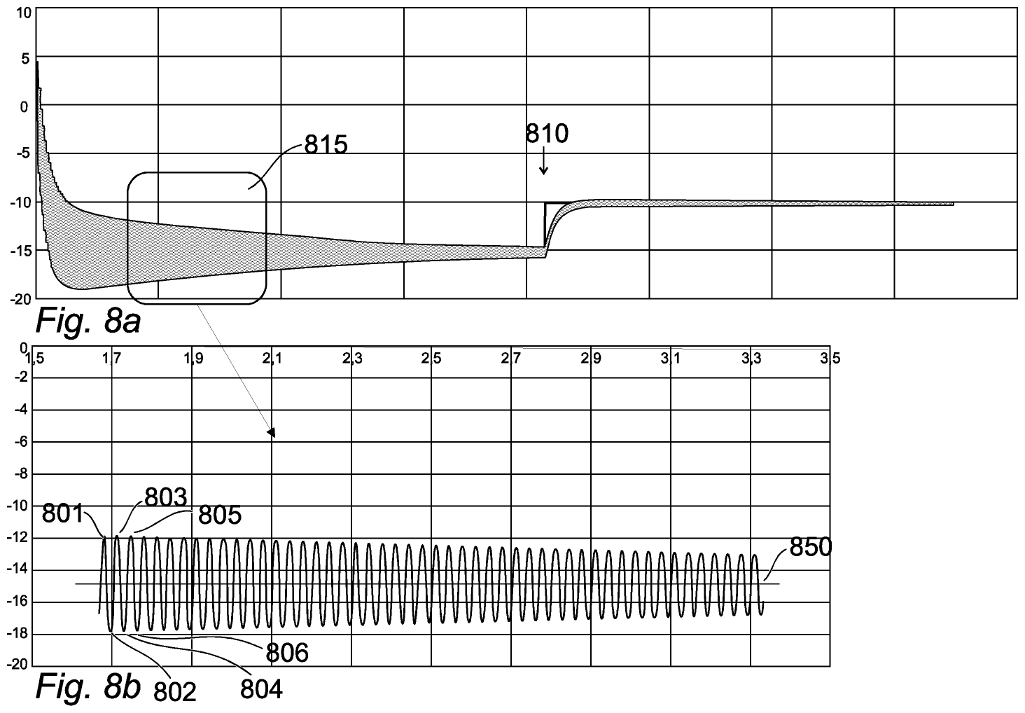

FIGS. 8a and 8b shows intrapleural pressure signal variation during inhalation and exhalation during respiration of a simulated human lung during a sped-up healing process. In FIG. 8a is shown the intrapleural pressure signal as a function of time. On the ordinate axis is pressure in cm H2O. On the abscissa axis is time. After a period of time 810, mean pressure can be seen regulated from minus 15 to minus 10. It can also be seen that during the healing process mean pressure is constant, while maximum and minimum pressures tend to get closer to the mean as the healing process advances with time. An area 815 is enlarged and shown in FIG. 8b to allow for study of individual breaths. Maximum 801, 803, 805 and minimum 802, 804, 806 pressures during individual breaths are shown. Mean pressure 850 is shown as almost horizontal line.
1. Drainage tube
2. Reference tube
3. Separation unit
4. Flexible tubing
5. Sensor module
6. Flow sensor
7. Filter
8. Pressure sensor
9. Positive relief valve
10. Peristaltic pump
11. Pump housing
12. Non-return valve
13. Reference pressure sensor
14. Fluid level sensor
15. Collection unit
16. (Spring loaded) connector
17. Memory chip
18. Accelerometer
19. Receiving connector
20. Atmospheric pressure sensor
21. Power and support unit

1. A drainage system for aspirating a drainage fluid from a body cavity of patient, the drainage system comprising:
a peristaltic pump for generating a suction pressure for aspirating the drainage fluid from the body cavity;
a fluid collection unit provided for collecting the drainage fluid;
a separator unit arranged to separate air from the drainage fluid arriving from the body cavity via tubing to the fluid collection unit;
wherein the peristaltic pump is configured to be connected to the patient and to the fluid collection unit via tubing;
a first pressure sensor arranged to sense the pressure in the body cavity;
a processor electrically connected to the first pressure sensor, and electrically connected to the pump, and configured to continuously collect pressure values from the first pressure sensor representative of the sensed pressure;
wherein the processor is configured to repeatedly calculate, based on collected pressure values, a minimum pressure representing a value of the minimum pressure sensed by the first pressure sensor during a predetermined period;
wherein the processor is configured to repeatedly calculate, based on collected pressure values, a maximum pressure representing a value of the maximum pressure sensed by the first pressure sensor during a predetermined period; and
wherein the drainage system is configured to visually present or otherwise communicate the minimum pressure and/or the maximum pressure or signals or values being calculated from them, to caretaking personnel or devices.

2. The drainage system according to claim 1, wherein the processor is configured to continually calculate a pressure difference Delta P, representing a difference between the maximum pressure and the minimum pressure.

3. The drainage system according to claim 2, wherein the pressure difference Delta P is presented on a display of the drainage system.

4. The drainage system according to claim 1, further comprising a second pressure sensor for sensing a second pressure value representing the pressure in the fluid collection unit.

5. The drainage system according to claim 1, wherein the processor is configured to calculate the minimum pressure as a mean value over a first predetermined time period.

6. The drainage system according to claim 1, wherein the processor is configured to calculate the minimum pressure as a mean value over a predetermined number of local minima or local maxima of a pressure value signal.

7. The drainage system according to claim 1, wherein the processor is configured to calculate the maximum pressure as a mean value over a first predetermined time period.

8. The drainage system according to claim 1, wherein the processor is configured to calculate the maximum pressure as a mean value over a predetermined number of local minima or local maxima of a pressure value signal.

9. The drainage system according to claim 1, wherein the drainage system is configured to measure a volume of the collected drainage fluid in the fluid collection unit.

10. The drainage system according to claim 1, wherein a determination of a level of separated air is performed by a disposable flow sensor inside a sensor module having a memory chip for logging of flow-data and configured to provide calibration data.

11. The drainage system according to claim 10, wherein the fluid collection unit is provided with an arrangement of valves for depressurizing the system while in a standby mode and while in an active mode;
wherein, in the stand by mode, air cannot pass the peristaltic pump and therefore is releasable to the atmosphere after passing the disposable flow sensor via a positive relief valve; and
wherein, in the active mode, the peristaltic pump will forward the air flow to a distal end of the flexible tubing and release the air flow to the atmosphere via a non-return valve provided with a virus- and bacterial filter to prevent contamination of a hospital environment.

12. The drainage system according to claim 11, wherein a fluid level sensor is a disposable screen-printed capacitive sensor connected to a pump housing for sending capacitive signals to the microcontroller for logging of drained fluid volumes in the fluid collection unit over time;
wherein the fluid collection unit has an arrangement of tooth-like projections that provide an individual spring force on each projection to create a spring-loaded contact between the fluid level sensor and a receiving connector in the pump housing.

13. The drainage system according to claim 9, wherein a pressure sensor is located inside the sensor module and configured to measure a volume of air entering the fluid collection unit by the alteration of pressure in the fluid collection unit, whereas an actual volume of a dead space is known with the aid of a fluid level sensor configured to measure volume of air entering the fluid collection unit over time.

14. The drainage system according to claim 13, wherein a reference sensor is configured to detect the patient's respiratory rate and a pressure variation, between inhalation and expiration to monitor the expansion of a collapsed lung as the pressure difference is decreasing in linearity to the lung expansion, the said-alteration in Delta P serving as an input to the processor to regulate a pump rate and thereof reduce or increase a negative pressure depending on an increase or decrease of the pressure variation.

15. The drainage system according to claim 12 being provided with an accelerometer for improving the accuracy to the measuring of fluid level in the fluid collection unit when the fluid level is not horizontal or tilted in relation to the fluid level sensor.

16. The drainage system according to claim 9, wherein the sensor module is provided with a memory chip for identification of the fluid collection unit;
wherein the memory chip is active in a standby mode and stores calibration data, pre-settings and saving data for being logged and transformable to another pump.

17. The drainage system according to claim 9, wherein the fluid collection unit is blow molded with polypropylene.

18. The drainage system according to claim 9, wherein the fluid collection unit is detachable from a pump housing for patient mobilization purpose and during that time being powered and data logged by a support unit.

19. A collection unit for collecting drainage fluid from a patient's body with the aid of a suction pressure, the collection unit comprising:
a container made of a pressure tight material, and rigid enough not to buckle when submitted to the suction pressure;
an inlet opening configured for connecting the collection unit to the patient's body in need of drainage;
an outlet opening configured to be connected to a source of suction pressure; and
a capacitive sensor including an electrically conductive film having a plurality of electrically separated elongated areas extending from a bottom of the collection unit and up to a maximum filling level of the collection unit.

20. The collection unit according to claim 19, wherein each elongated area extends further to a connector area that is arranged to adhere to a tooth-like springy portion of a polymer frame.
